(19) United States
(12) Patent Application Publication
(10) Pub. No.: US 2003/0176479 A1
(43) Pub. Date: Sep. 18, 2003

(54) Use of selective EP4 receptor agonists for the treatment of liver failure, loss of patency of the ductus arteriosus, glaucoma or ocular hypertension

(76) Inventors: Kimberly O. Cameron, East Lyme, CT (US); Bruce A. Lefker, Gales Ferry, CT (US)

Correspondence Address: PFIZER INC., PATENT DEPARTMENT, MS8260-1611, EASTERN POINT ROAD, GROTON, CT, 06340, US

(21) Appl. No.: 10/386,324

(22) Filed: Mar. 11, 2003

Related U.S. Application Data
(60) Provisional application No. 60/365,654, filed on Mar. 18, 2002.

(51) Int. Cl. A61K 31/41; A61K 31/4015

(52) U.S. Cl. 514/381; 514/424

(57) ABSTRACT

The present invention is directed to methods for treating liver failure, loss of patency of the ductus arteriosus, glaucoma or ocular hypertension, comprising administering to the patient in need thereof a therapeutically effective amount of a selective EP₄ receptor agonist of the Formulae

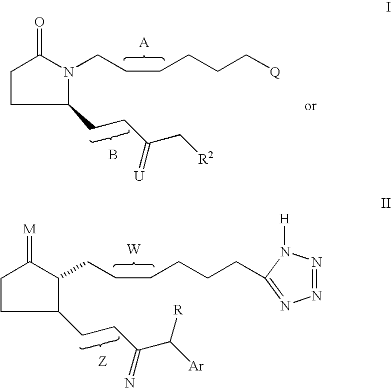

wherein the variables A, B, Q, ═U, and R² for Formula l; and the variables Ar, ═M, ═N, R, W, and Z for Formula II are as defined in the specification.

### FIELD OF THE INVENTION

The present invention relates to methods of using receptor selective prostaglandin (PGE₂) agonists for the treatment of liver failure, loss of patency of the ductus arteriosus, glaucoma or ocular hypertension in animals, particularly mammals. More specifically, the present invention relates to such methods using type 4 (EP₄) receptor selective prostaglandin (PGE₂) agonists.

### BACKGROUND OF THE INVENTION

The naturally occurring prostaglandins are comprised of several biological entities including PBD, PGE, PGF, PGG, PGH and PGI. It has been well documented that prostaglandins have effects on many of the organs and systems of the body. Prostaglandin E₂ (abbreviated as PGE₂ herein) is known to be a cyclooxygenase induced oxidative metabolite in the arachidonic acid cascade, and it has been well documented that prostaglandins, including PGE₂, have effects on many of the organs and systems of the body. For example, it is known that PGE₂ has cyto-protective activity, uterine contractile activity, a pain-inducing effect, a promoting effect on digestive peristalsis, an awakening effect, a sleep-inducing effect, a suppressive effect on gastric acid secretion, hypotensive activity and diuretic activity. In previous studies it has been found that the PGE₂ receptor has various subtypes, each possessing differing physiological roles. At this time, it is known that the PGE₂ receptor has four primary subtypes denoted EP₁, EP₂, EP₃ and EP₄, respectively, each of which mediates different effects in various tissues and cells (Coleman, R. A. et al., Pharm. Rev. 1994, 46(2), 205-229). The EP₄ receptor is distributed in such organs as the thymus, heart, kidney, liver, intestine, womb, ductus arteriosus and bone, and it is known that the EP₄ receptor is related to relaxation of smooth muscle, differentiation and proliferation of lymphocytes, proliferation of mesangial cells, and collagen production of the fibroblasts. In both the pig and the dog, modulation of the EP₄ receptor has been characterized with relaxation of the saphenous vein, and in the rabbit relaxation of the jugular vein occurs (Coleman, R. A. et al., Prostaglandins 1994, 47, 151).

Numerous studies have demonstrated the protective action of prostaglandin E₁ on experimental models of liver injury and on patients with fulminant viral hepatitis, with PGE₁ acting in many different ways to bring about this effect (Liu, X. L. et al. World J. Gastroenterol. 2000, 6(3), 326-329). For example, PGE₁ could act upon the PGE₁ receptor of diseased vessels to dilate them and increase portal venous flow, improve the microcirculation of the liver, clear the metabolites of the liver cells and increase oxygen supply to the liver tissues.

The EP₄ receptor is also expressed in the ductus arteriosus (Bhattacharya, M. et al., Circulation 1999, 100, 1751-1756). The ductus arteriosus is an arterial connection in the fetus, which directs blood away from the pulmonary circulation and towards the placenta where oxygenation occurs (Heymann, M. A.; Rudolph, A. M. Physiol. Rev. 1975, 55, 62-78). In one proposed model the EP₄ receptor in the ductus arteriosus acts as a sensor that responds to the perinatal drop in circulating levels of PGE₂ by triggering closure of the ductus arteriosus (Nguyen, M. et al., Nature 1997, 390, 78-81). Closure of the ductus arteriosus was observed in an in vivo fetal sheep model after administration of a selective EP₄ antagonist (PCT International Application WO 01/42281, published on Jun. 14, 2001). Maintaining the ductus arteriosus in the open, or patent state is desirable in the fetus and in infants with certain types of congenital heart defects where pulmonary or systemic blood flow depends on patency of the ductus arteriosus. Maintaining patency of the ductus arteriosus in infants with certain other types of congenital heart disease such as coarctation of the aorta, transposition of the great arteries, and Ebstein's anomaly may also be desirable. For example, infants with coarctation of the aorta, a condition constituting 7% to 8% of congenital cardiac defects, may have sudden onset of heart failure, cardiovascular collapse, and severe metabolic acidosis as the ductus arteriosus closes and distal perfusion is compromised. In cases such as these, PGE₁ infusions have been utilized to reopen and maintain the patency of the ductus arteriosus prior to surgical repair of the defect.

An excess of aqueous humor in the anterior chamber of the eye can result in elevated intraocular pressure or ocular hypertension. Ocular hypertension is a symptom and/or risk factor for glaucoma, a disease that can damage the optic nerve and cause blindness. The EP₄ receptor has been found in ocular tissues involved in the production of the aqueous humor, such as human ciliary epithelial cells and human ciliary muscle cells (Mukhopadhyay et al., Biochem. Pharmacol. 1997, 53, 1249-1255). Trabecular meshwork cells are known to be involved in the regulation of intraocular pressure (Clark et al., Investigative Opthalmology & Visual Science 1994, 35, 281-294; and Lutjen-Drescoll, Progress in Retinal and Eye Research 1998, 18, 91-119). The EP₄ receptor has also been found in human trabecular meshwork cells and it has been proposed that activation of the EP₄ receptors in the trabecular meshwork cells can result in relaxation of these cells, thereby lowering intraocular pressure (PCT International Patent Application WO 00/38667, published on Jul. 6, 2000).

As PGE₁ and PGE₂ bind to all four of the PGE₂ receptor subtypes (EP₁, EP₂, EP₃, and EP₄), various physiological activities may result, some of which may be an undesired side effect due to the lack of selectivity in binding to the PGE₂ receptor subtypes. Severe side effects have been associated with PGE₂ treatment. W. S. S. Jee, W. S. S. and Ma, Y. F. Bone, 1997, 21, 297-304.

Great Britain Patent Specification 1 553 595 discloses compounds of the formula

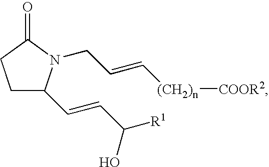

wherein the double bonds are cis or trans and the variables are defined as set forth therein. Those compounds are disclosed as having spasmogenic and spasmolytic activity, for example bronchodilatory and antihypertensive effects. The compounds are also disclosed as having utility in the inhibition of the secretion of gastric juice and as having abortive effects.

U.S. Pat. No. 4,115,401 discloses compounds of the formula

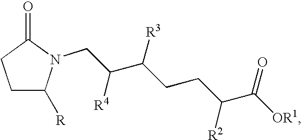

wherein the variables are defined as set forth therein. Those compounds are disclosed as having spasmogenic, cardiovascular and bronchodilatory effects.

U.S. Pat. No. 4,113,873 discloses compounds of the formula

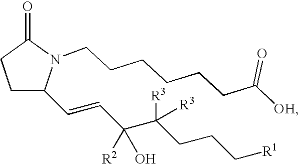

wherein the variables are defined as set forth therein. Those compounds are disclosed as having utility as a bronchodilator, as an antihypertensive agent, as an enhancer of spontaneous contraction of the uterus and for the treatment of gastro-intestinal disorders or gastric ulcers.

Great Britain Patent Specification 1 583 163 discloses compounds of the formula

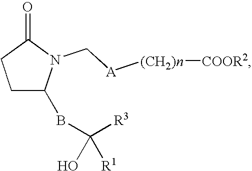

wherein the variables are defined as set forth therein. Those compounds are disclosed as having spasmogenic, bronchodilatory, vasoconstricting, vasodilating and abortive properties as well as utility in the inhibition of gastric acid secretion.

U.S. Pat. No. 4,177,346, discloses compounds of the formula

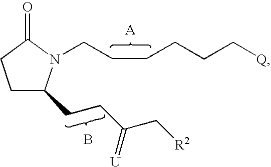

wherein the variables are defined as set forth therein. Those compounds are disclosed as having vasodilator, antihypertensive, bronchodilator, antifertility and antisecretory activity.

U.S. patent application Publication Nos. US 2001/0041729, which published on Nov. 15, 2001, and US 2001/0047105, which published on Nov. 29, 2001, disclose methods of treatment with compounds of the formula

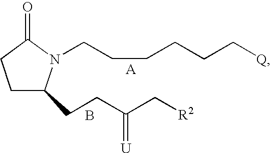

wherein the variables are defined as set forth therein. The methods of treatment disclosed in US 2001/0041729 include the treatment of acute or chronic renal failure or dysfunction, or a condition caused thereby, such as hypertension, congestive heart failure, glomerulonephritis, uremia or chronic renal insufficiency. The methods of treatment disclosed in US 2001/0047105 include the treatment of conditions which present with low bone mass, particularly osteoporosis, frailty, an osteoporotic fracture, a bone defect, childhood idiopathic bone loss, alveolar bone loss, mandibular bone loss, bone fracture, osteotomy, bone loss associated with periodontitis, or prosthetic ingrowth.

U.S. patent application Ser. No. 09/990,556, which was filed on Nov. 21, 2001, discloses compounds of the formula

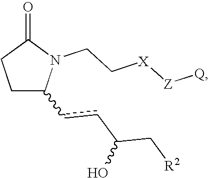

wherein the variables are as defined therein. The compounds are useful for the treatment of conditions which present with low bone mass such as osteoporosis, frailty, an osteoporotic fracture, a bone defect, childhood idiopathic bone loss, alveolar bone loss, mandibular bone loss, bone fracture, osteotomy, bone loss associated with periodontis, prosthetic ingrowth, or kidney dysfunction.

U.S. Pat. No. 3,932,389 provides 2-descarboxy-2-(tetrazol-5-yl)-11-desoxy-15-substituted-ω-pentanorprostaglandins with vasodilator activity, antihypertensive activity, bronchodilator activity, antifertility activity and antiulcer activity.

European Patent Application EP 1114816 discloses ω-substituted phenyl prostaglandin E derivatives useful for the treatment of immune diseases, asthma, abnormal bone formation, neurocyte death, pulmopathy, hepatopathy, sleeping disorders and platelet coagulations etc.

Certain 3,7-Dithiaprostanoic acid derivatives useful for treatment or prevention of immunologic diseases, asthma, abnormal bone formation, neuronal cell death, liver damage, nephritis, hypertension, myocardiac ischemia etc. are disclosed in U.S. Pat. Nos. 5,892,099 and 6,043,275.

PCT International Patent Application No. WO 99/02164 discloses methods and compositions for treating impotence or erectile dysfunction using prostaglandins that are selective EP₂ or EP₄ prostanoid receptor agonists.

Certain EP₂ receptor agonists, useful as agents for lowering intraocular pressure, have been disclosed in U.S. Pat. Nos. 5,462,968 and 5,698,598.

Certain prostaglandin E agonists useful for the treatment of glaucoma have been disclosed in PCT International Patent Application No. WO 00/38667, which published on Jul. 6, 2000.

### SUMMARY OF THE INVENTION

The present invention provides methods of treating liver failure, loss of patency of the ductus arteriosus, glaucoma or ocular hypertension in a mammal comprising administering to said mammal a selective EP₄ receptor agonist, an isomer thereof, a prodrug of said agonist or isomer, or a pharmaceutically acceptable salt of said agonist, isomer or prodrug. The selective EP₄ receptor agonists useful in the methods of the present invention are 1,5-disubstituted-2-pyrrolidones of Formula I or 2-descarboxy-2-(tetrazol-5-yl)-11-desoxy-15-substituted-ω-pentanor-prostaglandins of Formula II. The 1,5-disubstituted-2-pyrrolidone compounds of Formula I can be prepared as disclosed in U.S. Pat. No. 4,177,346, and U.S. patent application Publication US 2001/0047105, published on Nov. 29, 2001. The preparation of 2-descarboxy-2-(tetrazol-5-yl)-11-desoxy-15-substituted-ω-pentanor-prostaglandins of Formula II is described in U.S. Pat. No. 3,932,389.

A preferred group of the selective EP₄ receptor agonists for use in the methods of the present invention are compounds of Formula I:

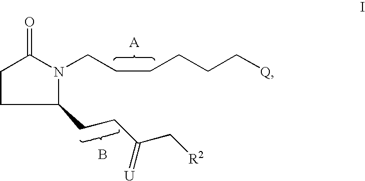

prodrugs thereof or pharmaceutically acceptable salts of said compounds or said prodrugs, wherein:

Q is COOR³, CONHR⁴ or tetrazol-5-yl;

A is a single or cis double bond;

B is a single or trans double bond;

═U is ═O,

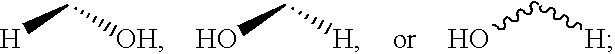

R² is α-thienyl, phenyl, phenoxy, monosubstituted phenyl or monosubstituted phenoxy, said substituents being selected from the group consisting of chloro, fluoro, phenyl, methoxy, trifluoromethyl and (C₁-C₃)alkyl;

R³ is hydrogen, (C₁-C₅)alkyl, phenyl or p-biphenyl;

R⁴ is COR⁵ or SO₂R⁵; and

R⁵ is phenyl or (C₁-C₅)alkyl.

A preferred group of selective EP₄ receptor agonists of Formula I are those compounds of Formula I wherein Q is 5-tetrazolyl. Particularly preferred compounds within this group include 5-(3-hydroxy-4-phenyl-but-1-enyl)-1-[6-(1H-tetrazol-5-yl)-hexyl]-pyrrolidin-2-one and 5-(3-hydroxy-4-phenyl-butyl)-1-[6-(1H-tetrazol-5-yl)-hexyl]-pyrrolidin-2-one.

Another preferred group of selective EP₄ receptor agonists of Formula I are those compounds of Formula I wherein Q is COOH. Particularly preferred compounds within this group include 7-[2-(3-hydroxy-4-phenyl-but-1-enyl)-5-oxo-pyrrolidin-1-yl]-heptanoic acid and 7-(2-(3-hydroxy-4-phenyl-butyl)-5-oxo-pyrrolidin-1-yl)-heptanoic acid.

Another preferred group of selective EP₄ receptor agonists for use in the methods of the present invention are compounds of Formula II:

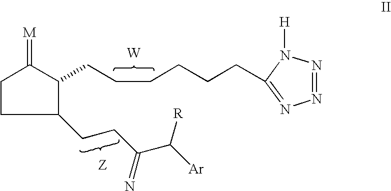

prodrugs thereof or pharmaceutically acceptable salts of said compounds or said prodrugs, wherein:

Ar is α- or β-thienyl, 5-phenyl-α- or β-thienyl, 5-lower alkyl-α- or β-thienyl, α- or β-napthyl tropyl, phenyl, 3,5-dimethylphenyl, 3,4-dimethoxyphenyl, 3,4-methylenedioxyphenyl, 3,4-dichlorophenyl, or mono-substituted phenyl wherein said substituent is bromo, chloro, fluoro, trifluoromethyl, phenyl, lower alkyl, or lower alkoxy;

R is hydrogen or methyl;

W is a single bond or cis double bond;

Z is a single bond or trans double bond; and

═M and ═N are each independently ═O,

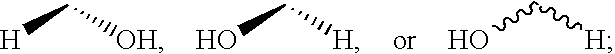

Another preferred group of selective EP₄ receptor agonists for use in the methods of the present invention are compounds of Formula II, wherein ═M and ═N are each ═O.

Another preferred group of selective EP₄ receptor agonists for use in the methods of the present invention are compounds of Formula II wherein

═M is

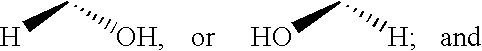

═N is ═O.

Another preferred group of selective EP₄ receptor agonists for use in the methods of the present invention are compounds of Formula II wherein

═M is

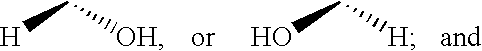

═N is

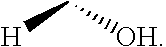

Yet another preferred group of selective EP₄ receptor agonists for use in the methods of the present invention are compounds of Formula II wherein ═M is ═O; and

═N is

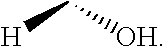

### DETAILED DESCRIPTION OF THE INVENTION

The term “treating”, “treat” or “treatment” as used herein includes preventative (e.g., prophylactic), palliative and curative treatment.

The term “pharmaceutically acceptable” means the carrier, vehicle, diluent, excipients, and/or salt must be compatible with the other ingredients of the formulation, and not deleterious to the patient.

The expression “prodrug” refers to a compounds that is a drug precursor which, following administration, releases the drug in vivo via some chemical or physiological process (e.g., a prodrug on reaching the physiological pH or through enzyme action is converted to the desired drug form). Exemplary prodrugs upon cleavage release the corresponding drug compounds.

The expression “pharmaceutically acceptable salt” refers to nontoxic anionic salts containing anions such as, but not limited to, chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as, but not limited to, sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine and tromethamine (2-amino-2-hydroxymethyl-1,3-propanediol).

The term “selective EP₄ receptor agonist” as used herein is a compound of Formula I or Formula II having a higher binding affinity for the EP₄ receptor than the EP₁, EP₂, and EP₃ receptors. A preferred group of the selective EP₄ receptor agonists are those compounds of Formulae I and II with an IC₅₀ at the EP₁, EP₂ and EP₃ receptor at least 10-fold greater than the IC₅₀ at the EP₄ receptor subtype. Accordingly, high selectivity or specificity for the EP₄ receptor, compared to other prostaglandin receptors, characterizes the compounds to be used in the methods of the present invention. Also, the receptor selectivity of the compounds to be used in the methods of the present invention results in the lessening or elimination of undesirable side effects caused by nonselective agents.

The methods of the present invention also include the use of isotopically-labeled compounds, which are identical to those recited in Formula I or Formula II, but for the fact that one or more atoms are replaced by an atom having an atomic mass or mass number different from the atomic mass or mass number usually found in nature. Examples of isotopes that can be incorporated into compounds of Formula I or Formula II include isotopes of hydrogen, carbon, nitrogen, oxygen, phosphorous, sulfur, fluorine and chlorine, such as ²H, ³H ¹³C, ¹⁴C, ¹⁵N, ¹⁸O, ¹⁷O, ³¹P, ³²P, ³⁵S, ¹⁸F and ³⁶Cl, respectively. Methods of treatment with compounds of Formula I or Formula II, prodrugs thereof, and pharmaceutically acceptable salts of said compounds and said prodrugs, and stereoisomers and diastereomeric mixtures of said compounds, prodrugs and salts, which contain the aforementioned isotopes and/or other isotopes of other atoms are within the scope of this invention. Certain isotopically-labeled compounds of Formula I or Formula II, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula I or Formula II and prodrugs thereof can generally be prepared by carrying out the procedures disclosed in U.S. Pat. No. 4,177,346, U.S. patent application Publication US 2001/0047105, published on Nov. 29, 2001 and U.S. Pat. No. 3,932,389, by substituting a readily available isotopically labeled reagent for a non-isotopically labeled reagent.

The compounds of Formula I or Formula II used in the methods of this invention have asymmetric carbon atoms, and therefore are enantiomers or diastereomers. Diasteromeric mixtures can be separated into their individual diastereomers on the basis of their physical chemical differences by methods known per se, for example, by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. Enantiomers and diastereomers of the compounds of Formula I or Formula II can also be prepared by utilizing suitable enantiomerically enriched starting materials, or by asymmetric or diastereoselective reactions to introduce asymmetric carbon atoms with the correct stereochemistry. All such isomers, including diastereomers, enantiomers and mixtures thereof are considered as compounds of Formula I or Formula II and can be used in the methods of this invention. Some of the compounds of Formula I or Formula II are acidic, and therefore, can form a salt with a pharmaceutically acceptable cation. All such salts are within the scope of the compounds of Formula I or Formula II, and can be prepared by conventional methods. For example, the salt can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

The selective EP₄ receptor agonists used in the methods of this invention can be adapted to therapeutic use in animals, e.g., mammals, and particularly humans. The utility of the selective EP₄ receptor agonists used in the methods of the present invention as medical agents in the treatment of liver failure, the loss of patency of the ductus arteriosus, glaucoma or ocular hypertension in animals, e.g., mammals, especially humans, is demonstrated by the activity of those agonists in conventional assays, including the EP₁, EP₂, EP₃, EP₄ receptor binding assay, the cyclic AMP assay, and can be demonstrated by activity in in vivo assays, including the liver failure model, all of which are described below. In vivo models, such as those described in U.S. Pat. Nos. 5,057,621, 5,462,968, and 5,698,598, can be used to demonstrate the ocular hypotensive effect of Formulae I and II compounds. Such assays also provide a means whereby the activities of the selective EP₄ receptor agonists can be compared to each other and with the activities of other known compounds and compositions. The results of these comparisons are useful for determining dosage levels in animals, e.g., mammals, including humans, for the treatment of such diseases.

Administration of a selective EP₄ receptor agonist according to the methods of this invention can be via any available mode that delivers the selective EP₄ receptor agonist systemically and/or locally (e.g. at the liver, ductus arteriosus, or eyes). These methods include oral routes, parenteral, intraduodenal routes, etc. Generally, the compounds of this invention are administered orally, but parenteral administration (e.g., intravenous, intramuscular, transdermal, subcutaneous, rectal or intramedullar) may be utilized, for example, where oral administration is inappropriate for the target or where the patient is unable to ingest the drug.

The methods of this invention are used for the treatment of liver failure, loss of patency of the ductus arteriosus, glaucoma, or ocular hypertension and can be carried out by either systemic or local application (e.g., to the ductus arteriosus, liver, or eyes) of the selective EP₄ receptor agonists. The selective EP₄ receptor agonists useful in the methods of the present invention are applied to the sites of the ductus arteriosus or liver, for example, either by injection of the compound in a suitable solvent, or in cases of open surgery, by local application thereto of the compound in a suitable vehicle, carrier or diluent. For administration to the eye, an ophthalmic preparation such as a gel, ointment, solution or suspension can be employed.

In any event, the amount and timing of the compound administered will be dependent on the patient being treated, on the severity of the affliction, on the manner of administration and on the judgment of the prescribing physician. Thus, because of patient to patient variability, the dosages given herein are a guideline and the physician may titrate doses of the drug compound to achieve the treatment (e.g., treat liver failure, loss of patency of the ductus arteriosus, glaucoma or ocular hypertension) that the physician considers appropriate for the patient. In considering the degree of treatment desired, the physician must balance a variety of factors such as age of the patient, body weight of the patient, symptom, presence of preexisting disease, desired therapeutic effect, the route of administration, and the duration of the treatment etc. In the human adult, the doses per person per dose are generally 1 μg to 100 mg, by oral administration, from once up to several times per day, and from 0.1 μg to 10 mg, by parenteral administration (preferably intravenously) from once up to several times per day, or by continuous administration for from 1 to 24 hours per day by intravenous infusion. For the treatment of neonates the dosage will have to be adjusted accordingly due to the patient's young age and low body weight. In general, in the methods of the present invention an amount of the selective EP₄ receptor agonist (compound of Formulae I and II) is used that is sufficient to treat liver failure, loss of patency of the ductus arteriosus, glaucoma or ocular hypertension. As the doses to be administered depend upon various conditions, there are cases in which doses lower or higher than the ranges specified above can be used.

The selective EP₄ receptor agonist compounds used in the methods of this invention are generally administered in the form of a pharmaceutical composition comprising at least one of the compounds of this invention together with a pharmaceutically acceptable vehicle or diluent. Thus, the selective EP₄ receptor agonist compound can be administered individually in any conventional form, such as oral, intranasal, parenteral, rectal or transdermal dosage form.

For oral administration the pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch, preferably potato or tapioca starch, and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compositions of this invention can be combined with various sweetening agents, flavoring agents, coloring agents, emulsifying agents and/or suspending agents, as well as such diluents as water, ethanol, propylene glycol, glycerin and various like combinations thereof.

The compounds can also be administered orally in solid solution with lipids such as cholesterol acetate. The inclusion of lipid in the formulation markedly increases absorption of the compound or analog. Preparation of such formulations is described in detail in Rudel, U.S. Pat. No. 3,828,106.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well known to those skilled in the art.

Compositions to be administered intravenously or by injection can be prepared as solutions of the compound in, for example, an isotonic aqueous solution, an alcohol solution, an ethanol-saline solution, or an ethanol-dextrose solution. Ethanol can be added to the solution to increase solubility and other additives such as methylparaben or other ingredients such as fillers, colorings, flavorings, diluents and the like can be included. The composition can also be administered as a suspension of the compound or analog in aqueous or non-aqueous media.

Among the preferred formulations for administration intravenously or by injection are complexes of the active ingredient with α-cyclodextrin. Preparation of complexes of compounds and analogs with α-cyclodextrin clathrates are described in detail in Hayashi et al., U.S. Pat. No. 4,054,736. Complexes wherein the ratio of α-cyclodextrin to a compound of this invention is 97:3 are especially preferred.

For purposes of transdermal (e.g.,topical) administration, dilute sterile, aqueous or partially aqueous solutions (usually in about 0.1% to 5% concentration), otherwise similar to the above parenteral solutions, are prepared.

For purposes of ophthalmic administration, an aqueous solution of the compound of Formula I or Formula II is generally preferred (typical concentration range is 0.001 to approximately 1% weight/volume). The aqueous solution can then be administered by instilling drops of the solution to the patient's eyes (usually 1 to 2 drops administered 1 to 4 times a day). For compounds of Formula I or Formula II with less water solubility, an aqueous suspension may be preferred. Other ophthalmic compositions known in the art, such as viscous or semi-viscous gels, or other types of solid or semi-solid compositions containing compounds of Formula I or Formula II may be employed.

The ophthalmic composition may also contain a preservative such as benzalkonium chloride, chlorobutanol, edetate disodium, phenylethyl alcohol, phenylmercuric acetate, phenyl mercuric nitrate, methyl paraben, propyl paraben, polyquaternium-1, sorbic acid, thimerosal, or other known preservatives (typical concentration range of the preservative is 0.001 to 1.0% weight/volume). A surfactant, such as Tween 80, can also be used in the ophthalmic composition. Various vehicles, such as polyvinyl alcohol, povidone, hydroxypropyl methyl cellulose, poloxamers, carboxymethyl cellulose, hydroxyethyl cellulose cyclodextrin and water can be used for the ophthalmic composition. The tonicity of the ophthalmic composition can be adjusted using a tonicity adjustor such as sodium chloride, potassium chloride, mannitol or glycerin. The ophthalmic composition can be buffered, preferably to a range of 4.5 to 8.0, using buffers such as acetate buffers, citrate buffers, phosphate buffers and borate buffers. The pH of the ophthalmic composition can be adjusted, preferably to a range between 4.5 to 8.0 using an appropriate acid or base. Antioxidants, such as sodium metabisulfite, sodium thiosulfate, acetylcysteine, butylated hydroxyanisole and butylated hydroxytoluene can also be used in the ophthalmic composition.

Methods of preparing various pharmaceutical compositions with a certain amount of active ingredient are known, or will be apparent in light of this disclosure, to those skilled in the art. For examples of methods of preparing pharmaceutical compositions, see Remington: The Science and Practice of Pharmacy, Alfonso R. Gennaro, Mack Publishing Company, Easton, Pa., 19th Edition (1995). Thus, as described above, the compounds of this invention may be administered to the patients in any of the known formulations or modes of administration.

Combination therapy can also be used in the methods of the present invention for the treatment of glaucoma or ocular hypertension. For the treatment of glaucoma or ocular hypertension, the selective EP₄ receptor agonists of Formula I or Formula II can be combined with other medicaments known to be useful for the treatment of glaucoma (anti-glaucoma agents), such as β-adrenergic blocking agents, carbonic anhydrase inhibitors, miotics and sympathomimetics. For example, β-adrenergic agents such as betaxolol, including its hydrochloride salt, and timolol, including its maleate salt can be combined with the selective EP₄ receptor agonists of Formula I or Formula II. Some examples of specific carbonic anhydrase inhibitors that can be used in combination with the selective EP₄ receptor agonists of Formula I or Formula II include brinzolamide, dichlorphenamide, and dorzolamide, including its hydrochloride salt. Miotics, such as demecarium bromide, can also be used in combination with the selective EP₄ receptor agonists of Formula I or Formula II. Sympathomimetics, such as brimonidine, including its tartrate salt, pheniramine, including its maleate salt, and phenylephrine, including its hydrochloride salt, can be used in combination with the selective EP₄ receptor agonists of Formula I or Formula II.

Advantageously, the present invention also provides kits for use by a consumer to treat liver failure, loss of patency of the ductus arteriosus, glaucoma or ocular hypertension. The kits comprise a) a pharmaceutical composition comprising a selective EP₄ receptor agonist (compound of Formula I or II); b) instructions describing methods of using the pharmaceutical compositions to treat liver failure, loss of patency of the ductus arteriosus, glaucoma or ocular hypertension; and c) a container. For methods of treating glaucoma or ocular hypertension the kit may also contain an anti-glaucoma agent as described above.

A “kit” as used in the instant application includes a container for containing the pharmaceutical compositions and may also include divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or other health care provider, or patient, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The documents cited herein, including any patents and patent applications, are hereby incorporated by reference.

### EXPERIMENTAL SECTION

In Vitro Assays

The compounds of Formula I or II, which are useful in the methods of the present invention, bind to the prostaglandin E₂ type 4 receptor (EP₄ receptor). The full-length coding sequence for the human EP₁ receptor is made in accordance with the procedure in Funk et al., Journal of Biological Chemistry, 1993, 268, 26767-26772. The full-length rat EP₂ receptor is made in accordance with the procedure in Nemoto et al., Prostaglandins and other Lipid Mediators, 1997, 54, 713-725. The full-length coding sequence for the human EP₃ receptor is made in accordance with the procedure in Regan et al., British Journal of Pharmacology, 1994, 112, 377-385. The full-length coding sequence for the rat EP₄ receptor is made in accordance with the procedure in Sando et al., Biochem. Biophys. Res. Comm. 1994, 200,1329-1333. These full-length receptors are used to prepare 293S cells expressing the human EP₁, rat EP₂, human EP₃ or rat EP₄ receptors.

Human EP₁, Rat EP₂, Human EP₃, Rat EP₄ Receptor Binding Assay

The full-length receptors described above are used to prepare 293S cells expressing the EP₁, EP₂, EP₃, and EP₄ receptors.

293S cells expressing either the human EP₁, rat EP₂, human EP₃ or rat EP₄ prostaglandin E₂ receptors are generated according to methods known to those skilled in the art. Typically, PCR (polymerase chain reaction) primers corresponding to the 5′ and 3′ ends of the published full length receptor are made according to the well known methods disclosed above and are used in an RT-PCR (reverse transcriptase-polymerase chain reaction) reaction using the total RNA from human kidney (for EP₁), rat kidney (for EP₂), human lung (for EP₃), or rat kidney (EP₄) as a source. PCR products are cloned by the TA overhang method into pCR2.1 (Invitrogen Corporation, Carlsbad, Calif.) and identity of the cloned receptor is confirmed by DNA sequencing. For expression of the rat EP₂ receptor, the confirmed cDNA is subcloned into the mammalian expression vector PURpCl, a vector generated by subcloning the selectable marker for puromycin resistance into the mammalian expression vector pCl (Promega, Madison, Wis.)

293S cells are transfected with either the cloned human EP₁ or EP₃ receptor in pcDNA3 by electroporation. Stable cell lines expressing either the human EP₁ or EP₃ receptor are established following selection of transfected cells with G418. 293S cells are transfected with the cloned rat EP₂ receptor in PURpCi by lipid mediated transfection. Stable cell lines expressing the rat EP₂ receptor are established following selection of transfected cells with puromycin. 293S cells are transfected with the cloned rat EP₄ receptor in pcDNA3 by lipid mediated transfection. Stable cell lines expressing the rat EP₄ receptor are established following selection of transfected cells with Geneticin® (Invitrogen, Carlsbad, Calif.).

Clonal cell lines expressing the maximal number of receptors are chosen following a whole cell ³H-PGE₂ binding assay using unlabeled PGE₂ as a competitor.

Membrane Preparation: All operations are performed at 4° C. Transfected cells expressing either prostaglandin E₂ type 1, type 2, type 3, or type 4 (EP₁, EP₂, EP₃, or EP₄, respectively) receptors are harvested and suspended to 2 million cells per ml in Buffer A [50 mM Tris-HCl (pH 7.4), 10 mM MgCI₂, 1 mM EDTA, 1 mM Pefabloc peptide, (Boehringer Mannheim Corp., Indianapolis, Ind.), 10 uM Phosporamidon peptide, (Sigma, St. Louis, Mo.), 1 uM pepstatin A peptide, (Sigma, St. Louis, Mo.), 10 uM elastatinal peptide, (Sigma, St. Louis, Mo.), 100 uM antipain peptide, (Sigma, St. Louis, Mo.)]. The cells are lysed by sonification with a Branson Sonifier (Branson Ultrasonics Corporation, Danbury, Conn.) in 2 fifteen-second bursts. Unlysed cells and debris are removed by centrifugation at 100 ×g for 10 min. Membranes are then harvested by centrifugation at 45,000×g for 30 minutes. Pelleted membranes are resuspended to 3-10 mg protein per ml, protein concentration being determined of the method of Bradford [Bradford, M., Anal. Biochem. 1976, 72, 248]. Resuspended membranes are then stored frozen at −80° C. until use.

Binding Assay: Frozen membranes prepared as above are thawed and diluted to 1 mg protein per ml in Buffer A above. 100 μl of the cell membrane preparation is combined with 5 μl of a solution of test compound of Formula I or II diluted in DMSO to a concentration 40 times the desired final concentration) and 95 μl of 3 nM ³H-prostaglandin E₂ (Amersham, Arlington Heights, Ill.) in Buffer A. The mixture (200 μL total volume) is incubated for 1 hour at 25° C. The membranes are then recovered by filtration through type GF/C glass fiber filters (Wallac, Gaithersburg, Md.) using a Tomtec harvester (Tomtec, Orange, Conn.). The membranes with bound ³H-prostaglandin E₂ are trapped by the filter, while the buffer and unbound ³H-prostaglandin E₂ pass through the filter into waste. Each sample is then washed 3 times with 3 ml of [50 mM Tris-HCl (pH 7.4), 10 mM MgCl₂, 1 mM EDTA]. The filters are then dried, by heating in a microwave oven. To determine the amount of ³H-prostaglandin bound to the membranes, the dried filters are placed into plastic bags with scintillation fluid and counted in a LKB 1205 Betaplate reader (Wallac, Gaithersburg, Md.). IC₅₀s are determined from the concentration of test compound required to displace 50% of the specifically bound ³H-prostaglandin E₂.

Determination of Cyclic AMP Elevation in 293S Cell Lines Stably Overexpressing Recombinant Rat EP₄ Receptors Assay

cDNA representing the complete open reading frame of the rat EP₄ receptor is generated by reverse transcriptase polymerase chain reaction using oligonucleotide primers based on published sequences. The full length coding sequence for the rat EP₄ receptor is made in accordance with the procedure in Sando et al., Biochem. Biophys. Res. Comm. 1994, 200, 1329-1333, and RNA from rat kidney (EP₄) as templates. 293S cells are transfected with the cloned rat EP₄ receptor in pcDNA3 by lipid mediated transfection. Stable cell lines expressing the rat EP₄ receptor are established following selection of transfected cells with Geneticin® (Invitrogen Corporation, Carlsbad, Calif.).

Clonal cell lines expressing the maximal number of receptors are chosen following a whole cell ³H-PGE₂ binding assay using unlabeled PGE₂ as a competitor. Transfectants demonstrating high levels of specific [³H]PGE₂ binding are further characterized by Scatchard analysis to determine Bₘₐₓ and K_{d}s for PGE₂. The lines selected for compound screening have approximately 256,400 receptors per cell and a K_{d}=2.9 nm for PGE₂ (EP₄). Constitutive expression of the receptor in parental 293-S cells is negligible. A stable cell line containing the rat EP₄ receptor is grown in Dulbecco's Mosified Eagle Medium/F12 (DMEM/F12) containing 10% fetal bovine serum and G418 (500 μg/ml) to 80% confluency.

cAMP responses in the 293-S/EP₄ lines are determined by detaching cells from culture flasks in 1 ml of calcium (Ca++) and magnesium (Mg++) deficient phosphate buffered saline (PBS) via vigorous pounding and then rinsing the cells with calcium (Ca++) and magnesium (Mg++) deficient phosphate buffered saline (PBS). The cells are resuspended in MEM (Minimum Essential Medium), 1% BSA (bovine serum albumin), 50 mM HEPES (N-[2-Hydroxyethyl]piperazine-N′-[2-ethanesulfonic acid]) at 37° C. The cell suspension is counted on a hemacytometer and diluted by adding MEM (Minimum Essential Medium) to a final concentration of 1×10⁶ cells/ml, and adding 3-isobutyl-1-methylxanthine (IBMX) to a final concentration of 1 mM. 200 microliters of cell suspension is immediately aliquoted into individual tubes and incubated for 10 minutes, uncovered, at 37° C., 5% CO₂, 95% relative humidity. The compound of Formula I or II to be tested in either dimethylsulfoxide (DMSO) or ethanol is then added to cells at 1:100 dilutions such that the final DMSO or ethanol concentration is 1%. Typically, the cells are treated with 6-8 different concentrations (in 1 log increments, such as those described below) of the compound of Formula I or II. Typical concentrations of the compound of Formula I or II in this assay are between 10⁻⁵M to 10⁻¹⁰M. For example, a six point compound dose response assay tests the compound of Formula I or II at concentrations of 10⁻⁵M, 10⁻⁶M, 10⁻⁷M, 10⁻⁸M, 10⁻⁹M and 10^{—10}M. Immediately after adding the test compound, the tubes are covered, mixed by inverting two times, and incubated at 37° C. for 12 minutes. Samples are then lysed by incubation at 100° C. for 10 minutes and immediately cooled on ice for 5 minutes to approximately 4° C. Cellular debris is pelleted by centrifugation at 3500×g for 5 minutes at approximately 4° C., and cleared lysates are transferred to fresh tubes. cAMP concentrations are determined using a commercially available ¹²⁵I-cAMP radioimmunoassay (RIA) kit (NEK-033, Perkin-Elmer Life Sciences, Inc., Boston, Mass.). The cleared lysates are diluted 1:100 in cAMP RIA assay buffer (included in kit) and centrifuged again. 50 microliters of the resulting supernatant is transferred to a 12×75 mm glass tube and data is collected by scintillation counting using a Wallac Cobra II Gamma Counter (Perkin-Elmer Wallac, Inc., Gaithersburg, Md.). EC₅₀ calculations are performed on a calculator using linear regression analysis on the linear portion of the dose response curves or using Data Fitter.

In Vivo Assays

The selective EP₄ receptor agonists of Formula I or Formula II can be evaluated in various in vivo liver failure models known in the art, such as an in vivo rat liver failure model (Kazuhiro, Kasai. et al., Gastroenterology 2001, 120 (Suppl. 1), A-541).

In Vivo Acute Liver Iniury Model

Methods: Acute liver failure in rats can be induced by intraperitoneal injection of one of carbon tetrachloride (CCl₄, 1 mg/kg), dimethylnitrosamine (DMN, 50 mg/kg), D-galactosamine (D-gal, 1 g/kg), or D-galactosamine with lipopolysaccharide (LPS), (D-gal, 1 g/kg; LPS 100 μg/kg). Immediately following the intaperitoneal injection of carbon tetrachloride, dimethylnitrosamine, D-galactosamine, or D-galactosamine with lipopolysaccharide, the test compound of Formula I or II or saline (as control) is administered. The test compound (a selective EP₄ receptor agonist of Formula I or II) can be administered at various doses such as 0.01, 0.05, 0.1 or 0.2 mg/kg. 24 hours after administration of the test compound of Formula I or II, the liver can be removed for histology and serum can be obtained for determination of total bilirubin (T-bil), aspartate aminotransferase (AST), and alanine aminotransferase (ALT). Massive hepatic necrosis with marked elevations in the levels of T-bil, AST, and ALT was observed in the saline treated control group. The effectiveness of the test compound in the above models can be determined by comparison of histology and serum results obtained for the animals treated with the test compound with the corresponding results from the saline control group.

### EXAMPLES

The examples presented herein are intended to illustrate particular embodiments of the invention, and are not intended to limit the specification or the claims in any manner.

### Examples 1-10

The in vitro Human EP₁, Rat EP₂, Human EP₃, Rat EP₄ Receptor Binding Assay and the Determination of cyclic AMP Elevation in 293S Cell Lines Stably Overexpressing Recombinant Rat EP₄ Receptors Assay, described hereinabove, were used to evaluate the following compounds. The compounds used in Examples 1-8 and 10 were prepared as described in U.S. patent application Publication US 2001/0047105, published on Nov. 29, 2001.

### Example 1

7-{2S-[4-(3-Chloro-phenyl)-3R-hydroxy-butyl]-5-oxo-pyrrolidin-1-yl}-heptanoic acid, prepared according to the procedure for Example 1 in U.S. patent application Publication US 2001/0047105, was found to have IC₅₀s of 22 nm (rat EP₄) and >3200 nm (rat EP₂, human EP₁, EP₃) in the binding assay, and an EC₅₀ of 8.8 nm in the cAMP (rat EP₄) elevation assay.

### Example 2

7-{2S-[3R-hydroxy-4-(3-trifluoromethyl-phenyl)-butyl]-5-oxo-pyrrolidin-1-yl}-heptanoic acid, prepared according to the procedure for Example 2 in U.S. patent application Publication US 2001/0047105, was found to have IC₅₀s of 21 nm (rat EP₄), 2760 nm (rat EP₂), and >3200 nm (human EP₁, EP₃), in the binding assay, and an EC₅₀ of 13.2 nm in the cAMP (rat EP₄) elevation assay.

### Example 3

5S-[4-(3-Chloro-phenyl)-3-hydroxy-butyl]-1-[6-(2H-tetrazol-5-yl)-hexyl]-pyrrolidin-2-one, prepared according to the procedure for Example 3 in U.S. patent application Publication US 2001/0047105, was found to have IC₅₀s of 38 nm (rat EP₄), 2370 nm (rat EP₂), and >3200 nm (human EP₁, EP₃), in the binding assay, and an EC₅₀ of 33.1 nm in the cAMP (rat EP₄) elevation assay.

### Example 4

5S-[3R-Hydroxy-4-(3-trifluoromethyl-phenyl)-butyl]-1-[6-(2H-tetrazol-5-yl)-hexyl]-pyrrolidin-2-one, prepared according to the procedure for Example 4 in U.S. patent application Publication US 2001/0047105, was found to have IC₅₀s of 33 nm (rat EP₄), and >3200 nm (rat EP₂, human EP₁, EP₃), in the binding assay, and an EC₅₀ of 70.2 nm in the cAMP (rat EP₄) elevation assay.

### Example 5

5-[4-(4-Fluoro-phenyl)-3-hydroxy-butyl]-1-[6-(2H-tetrazol-5-yl)-hexyl]-pyrrolidin-2-one, prepared according to the procedure for Example 5 in U.S. patent application Publication US 2001/0047105, was found to have IC₅₀s of 508 nm (rat EP₄), and >3200 nm (rat EP₂, human EP₁, EP₃), in the binding assay.

### Example 6

5-(4-Biphenyl-3-yl-3-hydroxy-butyl)-1-[6-(2H-tetrazol-5-yl)-hexyl]-pyrrolidin-2-one, prepared according to the procedure for Example 6 in U.S. patent application Publication US 2001/0047105, was found to have IC₅₀s of 50 nm (rat EP₄), 3050 nm (rat EP₂) and >3200 nm (human EP₁, EP₃), in the binding assay, and an EC₅₀ of 175 nm in the cAMP (rat EP₄) elevation assay.

### Example 7

5-[4-(3-Fluoro-phenyl)-3-hydroxy-butyl]-1-[6-(2H-tetrazol-5-yl)-hexyl]-pyrrolidin-2-one, prepared according to the procedure for Example 7 in U.S. patent application Publication US 2001/0047105, was found to have IC₅₀s of 96 nm (rat EP₄), and >3200 nm (rat EP₂), in the binding assay, and an EC₅₀ of 200 nm in the cAMP (rat EP₄) elevation assay.

### Example 8

5S-[4-(3-Chloro-phenyl)-3R-hydroxy-butyl]-1-[6-(2H-tetrazol-5-yl)-hexyl]-pyrrolidin-2-one, prepared according to the procedure for Example 8 in U.S. patent application Publication US 2001/0047105, was found to have IC₅₀s of 28 nm (rat EP₄), and >3200 nm (rat EP₂), in the binding assay, and an EC₅₀ of 24.6 nm in the cAMP (rat EP₄) elevation assay.

### Example 9

7-(2-(3-hydroxy-4-phenyl-butyl)-5-oxo-pyrrolidin-1-yl)-heptanoic acid was found to have IC₅₀s of 54 nm (rat EP₄), and >3200 nm (rat EP₂, human EP₁, EP₃), in the binding assay, and an EC₅₀ of 32.5 nm in the cAMP (rat EP₄) elevation assay.

### Example 10

7-{2S-[3-Hydroxy-4-(3-phenoxy-phenyl)-butyl]-5-oxo-pyrrolidin-1-yl}-heptanoic acid, prepared according to the procedure for Example 10 in U.S. patent application Publication US 2001/0047105, was found to have IC₅₀s of 536 nm (rat EP₄), and >3200 nm (rat EP₂), in the binding assay.

What is claimed is:

1. A method of treating liver failure, loss of patency of the ductus arteriosus, glaucoma or ocular hypertension, comprising administering to a patient in need thereof a compound of Formula I: 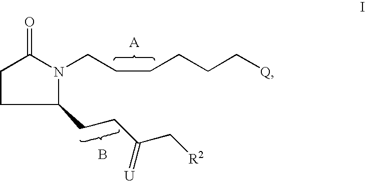
a prodrug thereof, or a pharmaceutically acceptable salt of the compound or prodrug, wherein:
Q is COOR³, CONHR⁴ or tetrazol-5-yl;
A is a single or cis double bond;
B is a single or trans double bond;
═U is ═O, 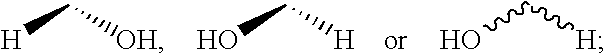
R² is α-thienyl, phenyl, phenoxy, monosubstituted phenyl or monosubstituted phenoxy, said substituents being selected from the group consisting of chloro, fluoro, phenyl, methoxy, trifluoromethyl and (C₁-C₃)alkyl;
R³ is hydrogen, (C₁-C₅)alkyl, phenyl or p-biphenyl;
R⁴ is COR⁵ or SO₂R⁵; and
R⁵ is phenyl or (C₁-C₅)alkyl.

2. A method of claim 1, wherein Q is 5-tetrazolyl.

3. A method of claim 2, wherein the compound of Formula I is
5-(3-Hydroxy-4-phenyl-but-1-enyl)-1-[6-(1H-tetrazol-5-yl)-hexyl]-pyrrolidin-2-one,
5-(3-Hydroxy-4-phenyl-butyl)-1-[6-(1H-tetrazol-5-yl)-hexyl]-pyrrolidin-2-one,
5S-[4-(3-Chloro-phenyl)-3-hydroxy-butyl]-1-[6-(2H-tetrazol-5-yl)-hexyl]-pyrrolidin-2-one,
5S-[4-(3-Chloro-phenyl)-3R-hydroxy-butyl]-1-[6-(2H-tetrazol-5-yl)-hexyl]-pyrrolidin-2-one,
5S-[3R-Hydroxy-4-(3-trifluoromethyl-phenyl)-butyl]-1-[6-(2H-tetrazol-5-yl)-hexyl]-pyrrolidin-2-one,
5-[4-(4-Fluoro-phenyl)-3-hydroxy-butyl]-1-[6-(2H-tetrazol-5-yl)-hexyl]-pyrrolidin-2-one,
5-(4-Biphenyl-3-yl-3-hydroxy-butyl)-1-[6-(2H-tetrazol-5-yl)-hexyl]-pyrrolidin-2-one, or
5-[4-(3-Fluoro-phenyl)-3-hydroxy-butyl]-1-[6-(2H-tetrazol-5-yl)-hexyl]-pyrrolidin-2-one.

4. A method of claim 1, wherein Q is COOH.

5. A method of claim 4, wherein the compound of Formula I is
7-(2-(3-Hydroxy-4-phenyl-butyl)-5-oxo-pyrrolidin-1-yl)-heptanoic acid,
7-[2-(3-Hydroxy-4-phenyl-but-1-enyl)-5-oxo-pyrrolidin-1-yl]-heptanoic acid,
7-{2S-[4-(3-Chloro-phenyl)-3R-hydroxy-butyl]-5-oxo-pyrrolidin-1-yl}-heptanoic acid,
7-{2S-[3R-Hydroxy-4-(3-trifluoromethyl-phenyl)-butyl]-5-oxo-pyrrolidin-1-yl}-heptanoic acid, or
7-{2S-[3-Hydroxy-4-(3-phenoxy-phenyl)-butyl]-5-oxo-pyrrolidin-1-yl}-heptanoic acid.

6. A method of treating liver failure, loss of patency of the ductus arteriosus, glaucoma or ocular hypertension, comprising administering to a patient in need thereof a compound of Formula II: 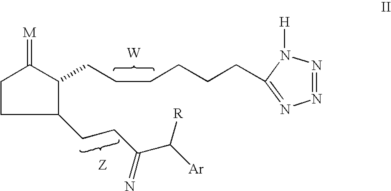
a prodrug thereof, or a pharmaceutically acceptable salt of the compound or prodrug, wherein:
Ar is α- or β-thienyl, 5-phenyl-α- or β-thienyl, 5-lower alkyl-α- or β-thienyl, α- or β-napthyl, tropyl, phenyl, 3,5-dimethylphenyl, 3,4-dimethoxyphenyl, 3,4-methylenedioxyphenyl, 3,4-dichlorophenyl, or mono-substituted phenyl wherein said substituent is bromo, chloro, fluoro, trifluoromethyl, phenyl, lower alkyl, or lower alkoxy;
R is hydrogen or methyl;
W is a single bond or cis double bond;
Z is a single bond or trans double bond; and
═M and ═N are each independently ═O,

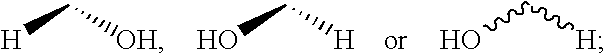

7. A method of claim 6, wherein ═M and ═N are each ═O.

8. A method of claim 6, wherein ═M is 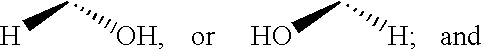
═N is ═O.

9. A method of claim 6, wherein ═M is 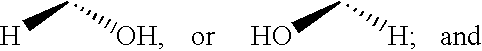
═N is

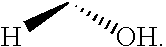

10. A method of claim 6, wherein ═M is ═O; and
═N is

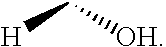

11. The method of claim 1, wherein the method is the treatment of liver failure.

12. The method of claim 1, wherein the method is the treatment of the loss of patency of the ductus arteriosus.

13. The method of claim 1, wherein the method is the treatment of glaucoma or ocular hypertension.

14. The method of claim 6, wherein the method is the treatment of liver failure.

15. The method of claim 6, wherein the method is the treatment of the loss of patency of the ductus arteriosus.

16. The method of claim 6, wherein the method is the treatment of glaucoma or ocular hypertension.
